(19) United States
(12) Patent Application Publication
(10) Pub. No.: US 2018/0168933 A1
(43) Pub. Date: Jun. 21, 2018

(54) Diclofenac Dosage Forms

(71) Applicant: Apotex Technologies Inc., Toronto (CA)

(72) Inventor: Bernard Charles Sherman, Toronto (CA)

(21) Appl. No.: 15/848,754

(22) Filed: Dec. 20, 2017

Related U.S. Application Data
(60) Provisional application No. 62/436,572, filed on Dec. 20, 2016.

(51) Int. Cl. A61J 3/07; A61J 3/00; A61K 9/48; A61P 29/00

(52) U.S. Cl. CPC A61J 3/07; A61J 3/005; A61K 9/4866; A61P 29/00; A61K 9/4858; A61K 9/4891; A61K 9/485

(57) ABSTRACT

The present invention relates to oral dosage forms of diclofenac, and processes for their manufacture, that provide a rapidly absorbed dose of diclofenac without requiring milling of diclofenac to submicron sizes.

### CROSS-REFERENCE TO RELATED APPLICATION

This application claims benefit of U.S. Provisional Patent Application No. 62/436,572, filed Dec. 20, 2016, the disclosure of which is hereby incorporated in its entirety by reference.

### TECHNICAL FIELD

The present invention relates to oral dosage forms of diclofenac and processes for their manufacture.

### BACKGROUND

Diclofenac is a non-steroidal anti-inflammatory drug (“NSAID”) that is used to treat various forms of pain, including the management of mild to moderate pain, osteoarthritic pain and the pain associated with rheumatoid arthritis. Various dosage forms of diclofenac, including capsules and tablets, comprising diclofenac or a salt thereof have been available for many years.

Because of potential adverse effects, products containing diclofenac or a salt thereof are recommended to be used at the lowest effective dose consistent with individual patient treatment goals. Further, since diclofenac is used in the treatment of pain, it is desirable for a diclofenac dosage form for oral administration (e.g., a capsule of tablet) to exhibit absorption that is rapid, and which provides the desired peak plasma concentration (Cₘₐₓ) with the lowest possible dose.

Recently, a new, low dose, formulation of diclofenac has been introduced comprising capsules containing diclofenac in strengths of 18 mg and 35 mg under the tradename ZORVOLEX™. ZORVOLEX™ capsules achieve rapid absorption of the diclofenac content because the diclofenac within the capsules is provided in the form of submicron-sized particles. Details relating to the complex manufacturing process required to provide this rapid absorption dosage form are provided in U.S. Pat. No. 8,679,544.

A major drawback to the process of reducing particles to submicron size is the inherent expense of the milling steps involved in preparing submicron-sized materials, as well the difficulties that can be involved in handling and formulating submicron-sized materials.

As a result, there remains a need in the art to provide diclofenac dosage forms capable of providing low doses of diclofenac with rapid absorption, and in particular, dosage forms that provide equivalent exposure to that obtained with ZORVOLEX™ capsules, but which can be manufactured by a simplified process that does not require milling steps in order to prepare submicron-sized particles of diclofenac.

### SUMMARY

The present invention provides oral dosage forms of diclofenac, and processes for the production of oral dosage forms of diclofenac, that are capable of providing rapidly absorbed doses, without requiring the milling of diclofenac particles to submicron sizes. Preferred embodiments of the invention include capsules that provide equivalent AUC_{T} and Cₘₐₓ levels of diclofenac to those provided by an equivalent dose of ZORVOLEX™ capsules.

In a first aspect of the present invention, there is provided a process of manufacture of a solid dosage form of diclofenac for oral administration comprising the steps of:
(i) forming a solution by dissolving diclofenac and a basic sodium or potassium compound in a solvent comprising water, a volatile organic solvent, or a mixture thereof;
(ii) applying the solution to a solid substrate comprising one or more pharmaceutically acceptable excipients to form a mixture;
(iii) forming a dried mixture by evaporating the solvent from the mixture; and
(iv) further processing the dried mixture into a dosage form, optionally with one or more additional pharmaceutically acceptable excipients.

In a preferred embodiment of the first aspect, the basic sodium or potassium compound is sodium hydroxide or potassium hydroxide, and is more preferably sodium hydroxide. In another preferred embodiment of the first aspect, the solvent comprises water, an alcohol, or a mixture thereof; more preferably, the solvent comprises water or an alcohol, and is most preferably methanol.

In another preferred embodiment of the first aspect, the solution is applied to the solid substrate in a granulator, and the resulting mixture is a wet granulate. Preferably, the granulation is conducted by applying the solution to the substrate at an elevated temperature, preferably in the range of about 55° C. to about 70° C. In another preferred embodiment of the first aspect, the further processing in step (iv) comprises deagglomerating the dried mixture or granulate prior to mixing this material with one or more pharmaceutically acceptable excipients.

In another preferred embodiment of the first aspect, the substrate comprises a disintegrant, a carbonate, or a mixture thereof. Preferably, the disintegrant is selected from starches, modified starches, celluloses, modified celluloses, carmellose calcium, croscarmellose sodium, crospovidone, and mixtures thereof. Most preferably, the disintegrant is croscarmellose sodium. Preferably, the carbonate is calcium carbonate. In a further preferred embodiment, the substrate is a mixture of a disintegrant and a carbonate, and most preferably, a mixture of croscarmellose sodium and calcium carbonate.

In another preferred embodiment of the first aspect, the process manufactures a solid dosage form of diclofenac that provides equivalent (the ratios of the mean values of the test and reference formulation are within the range of 80% to 125%) AUC_{T} and Cₘₐₓ levels to those provided by ZORVOLEX™ capsules.

In a second aspect of the present invention, there is provided a solid dosage form for the oral administration of diclofenac prepared by:
(i) forming a solution by dissolving diclofenac and a basic sodium or potassium compound in a solvent comprising water, a volatile organic solvent, or a mixture thereof;
(ii) mixing the solution with a solid substrate comprising one or more pharmaceutically acceptable excipients to form a mixture;
(iii) forming a dried mixture by evaporating the solvent from the mixture; and
(iv) further processing the dried mixture into a dosage form, optionally with one or more additional pharmaceutically acceptable excipients.

In a preferred embodiment of the second aspect, the solution is applied to the solid substrate in a granulator, and the resulting mixture is a wet granulate. Preferably, the granulation is conducted by applying the solution to the substrate at an elevated temperature, preferably in the range of about 55° C. to about 70° C. In a further preferred embodiment of the second aspect, the dried mixture or granulate is deagglomerated prior to mixing with one or more pharmaceutically acceptable excipients.

In a further preferred embodiment of the second aspect, the substrate in the dried mixture comprises a disintegrant and a carbonate, and the dried mixture is mixed with a lubricant and a glidant. More preferably, the dosage form is a capsule filled with a granulated dried mixture comprising diclofenac sodium and a substrate comprised of a mixture of croscarmellose sodium and calcium carbonate, and the granulated dried mixture is mixed with magnesium stearate and colloidal silicon dioxide.

In another preferred embodiment of the second aspect, the solid dosage form provides equivalent AUC_{T} and Cₘₐₓ levels to those provided by ZORVOLEX™ capsules.

In a third aspect of the present invention, there is provided an orally-administrable solid dosage form of diclofenac comprising a substrate coated with a sodium or potassium diclofenac salt, and optionally also comprising, one or more pharmaceutically acceptable excipients.

In a preferred embodiment of the third aspect, the substrate is a disintegrant, a carbonate, or a mixture thereof. More preferably, the substrate is a mixture of croscarmellose sodium and calcium carbonate.

In another preferred embodiment of the third aspect, the solid dosage form comprises capsules filled with diclofenac sodium coated onto a solid substrate comprising a mixture of croscarmellose sodium and calcium carbonate, magnesium stearate and colloidal silicon dioxide.

In another preferred embodiment of the third aspect, the solid dosage form provides equivalent AUC_{T} and Cₘₐₓ levels to those provided by ZORVOLEX™ capsules.

In a fourth aspect of the present invention, there is provided an orally-administrable solid dosage form of diclofenac comprising a first and second dose of diclofenac, wherein the first dose of diclofenac is an amount of a dried mixture of diclofenac sodium or potassium on a solid substrate to provide a first, rapidly absorbed, dose of diclofenac, and the second dose of diclofenac is an amount of diclofenac, or a pharmaceutically acceptable salt thereof, in the form of extended release granules, pellets or mini-tablets.

Other aspects and features of the present invention will become apparent to those ordinarily skilled in the art upon review of the following description of specific embodiments of the invention.

### DETAILED DESCRIPTION

The present invention provides oral dosage forms of diclofenac comprising one or more pharmaceutical excipients coated with a sodium or potassium salt of diclofenac, optionally mixed with one or more additional pharmaceutically acceptable excipients. Through the use of the dosage forms of the present invention, it is possible to provide doses of diclofenac that are rapidly absorbed following administration without requiring the milling of diclofenac to submicron sizes during the manufacturing process. In preferred embodiments of the present invention, there is provided a dosage form that provides equivalent AUC_{T} and Cₘₐₓ levels of diclofenac to those provided by an equivalent dose of ZORVOLEX™ capsules without requiring the use of diclofenac that has been milled to submicron sizes.

In one embodiment of the present invention, a process is provided for the manufacture of solid dosage forms, preferably capsules or tablets, for oral administration comprising the steps of:
(i) forming a solution by dissolving diclofenac and a basic sodium or potassium compound in a solvent comprising water, a volatile organic solvent, or a mixture thereof;
(ii) applying the solution to a solid substrate comprising one or more pharmaceutically acceptable excipients to form a mixture;
(iii) forming a dried mixture by evaporating the solvent from the mixture; and
(iv) further processing the dried mixture into a dosage form, optionally with one or more additional pharmaceutically acceptable excipients.

Suitable basic sodium or potassium compounds for use with the process of the present invention are those capable of forming a sodium or basic salt with diclofenac. Preferably, the basic sodium or potassium compound is selected from sodium hydroxide and potassium hydroxide.

Within the present invention, the substrate to which the solution of diclofenac is applied is a pharmaceutical excipient. Preferably, the substrate to which the solution of diclofenac is applied comprises a disintegrant, a carbonate, or a mixture thereof.

Suitable disintegrants for use within the present invention are those excipients that cause or facilitate breakup of the contents of a dosage form when it comes in contact with liquid. In preferred embodiments, the disintegrant is an excipient that is insoluble in water, but swells when wetted to cause disintegration. Examples of preferred disintegrants for use with the present invention include starches, modified starches, cellulose, modified cellulose, carmellose calcium, croscarmellose sodium, crospovidone, or combinations thereof. A particularly preferred disintegrant is croscarmellose sodium.

While any pharmaceutically acceptable carbonate may be used with the process of the present invention, the carbonate is preferably calcium carbonate.

Suitable solvents for use with the processes of the present invention include water and volatile organic solvents. Preferably, the volatile organic solvent is an alcohol, and is most preferably methanol.

When applying the solution containing diclofenac and the basic sodium or potassium compound to the solid substrate, the substrate and solution can be mixed together, for example, in a standard wet granulation process, or, alternatively, the solution can be applied onto the substrate using other standard methods known to one skilled in the art, such as spray-coating.

Preferably, the solution is applied to the solid substrate by mixing the solution and solid substrate in a wet granulation process, and then removing the solvent by drying the mixture, thereby allowing the solvent to evaporate. Preferably, the wet granulation process is carried out by applying the solution to the substrate at an elevated temperature, preferably in the range of 55° C. to 70° C.

Evaporation of the solvent through spray-coating or conventional drying provides a substrate that is coated with a sodium or potassium salt of diclofenac in the form of a dried mixture. This dried mixture can then be further processed into a solid dosage form, such as a capsule or tablet, using common methods of manufacture that would be known to one of skilled in the art.

Suitable pharmaceutical excipients are those commonly known in the art for the preparation of immediate release dosage forms, and in particular, capsules and tablets, and may be selected from diluents, binders, glidants, disintegrants and lubricants. These excipients and their common methods of use are well-known to the skilled person and are described in common texts, such as The Handbook of Pharmaceutical Excipients and Remington The Science and Practice of Pharmacy. Preferred diluents are selected from dicalcium phosphate, calcium sulfate, lactose, cellulose, mannitol, starch and powdered sugar. Preferred binders are selected from starches, gelatin, sugars, cellulose polymers and polyvinylpyrrolidone. Preferred glidants are selected from colloidal silicon dioxide and talc. Preferred disintegrants are selected from those described above for use as a solid substrate. Preferred lubricants are selected from talc, magnesium stearate, calcium stearate and polyethylene glycol. Optionally, other pharmaceutical excipients, such as coloring agents, can be used.

Further processing of the dried mixture can involve, for example, deagglomeration. When the further processing step involves deagglomeration, the screen used is suitable for removal of large agglomerates (for example, particles greater than 1,000 μm). The dried mixture, with or without further processing, is optionally mixed with one or more pharmaceutically acceptable excipients in the preparation of the oral dosage form, which is preferably a capsule or tablet, and most preferably, a capsule. Capsules can be prepared, for example, by filling capsules with the dried mixture, optionally, with one or more pharmaceutically acceptable excipients. Alternatively, the dried mixture is used in the preparation of mini-tablets, optionally with one or more pharmaceutically acceptable excipients, which are then filled into capsules. Tablets can be prepared, for example, by direct compression, dry granulation or wet granulation of the dried mixture with one or more additional pharmaceutically acceptable excipients. When wet granulation is used, it is preferred that the solvent used does not lead to dissolution of the diclofenac and its salt from the solid substrate. These methods of preparing solid oral dosage forms are well known to the skilled person, and are described in common texts, such as Remington The Science and Practice of Pharmacy.

Optionally, immediate release coatings can be applied to either the dried mixture, granules formed from the dried mixture and other pharmaceutically acceptable excipients, mini-tablets or tablets. Suitable immediate release coatings (i.e., coatings not intended to delay the release of diclofenac for the formulation) are well known to those skilled in the art, and are described, for example, in common texts, such as Remington The Science and Practice of Pharmacy.

In a preferred embodiment of the invention, the dried mixture is agglomerated prior to being mixed with a lubricant and a glidant. Preferably, the lubricant is magnesium stearate and the glidant is colloidal silicon dioxide. After mixing the deagglomerated dried mixture with these excipients, the mixture is filled into capsules to provide a dosage form having 18 mg or 35 mg diclofenac.

In a further preferred embodiment of the present invention, the oral dosage form provides equivalent AUC_{T} and Cₘₐₓ levels to those provided by ZORVOLEX™ capsules.

AUC_{T} as used herein is defined as the area under the curve of serum concentration versus time for a chosen period of time after ingestion, such as, for example 24 hours. AUC_{T} ratio as used herein is defined as the ratio of mean AUC_{T} provided by the test product to the mean AUC_{T} provided by the reference product.

Cₘₐₓ as used herein is defined as the peak serum concentration. Cₘₐₓ ratio as used herein is defined as the ratio of mean Cₘₐₓ provided by the test product to the mean Cₘₐₓ provided by the reference product.

Oral dosage forms of the present invention are considered to provide equivalent AUC_{T} and Cₘₐₓ levels to those provided by ZORVOLEX™ capsules if the Cₘₐₓ and AUC_{T} ratios are within the range of 80% to 125%.

In a further embodiment of the invention, oral dosage forms are provided comprising a combination of a first and second dose of diclofenac. The first dose of diclofenac is an amount of a dried mixture of diclofenac sodium or potassium coated on a solid substrate to provide a first, rapidly absorbed, dose of diclofenac. If desired, the dried mixture is further processed, for example, by wet or dry granulation with one or more additional pharmaceutically acceptable excipients, before being combined with the second dose of diclofenac to prepare the oral dosage form. The second dose of diclofenac is an amount of diclofenac, or a pharmaceutically acceptable salt thereof, in the form of extended release granules, pellets or mini-tablets.

The extended release character of the second dose of diclofenac can be provided through the use of extended release matrices or extended release coatings. If desired, delayed release coatings can also be used to provide a delayed release of the second dose of diclofenac such that the second dose provides either delayed release followed by extended release, or delayed release followed by immediate release. Excipients known to provide extended and delayed release, as well as the preparation of extended and delayed release dosage forms are well known to the skilled person, and are described, for example, in common texts, such as The Handbook of Pharmaceutical Excipients and Remington The Science and Practice of Pharmacy.

### EXAMPLES

The following examples are illustrative of the aspects and embodiments of the invention described herein. These examples should not be considered to limit the spirit or scope of the invention in any way.

### Example 1

7.96 kilos of diclofenac and 1.1 kilos of sodium hydroxide were dissolved in 13.65 kilos of methanol to form a solution. The solution was blended into a mixture of 22.8 kilos of croscarmellose sodium (a disintegrant) and 7.59 kilos of calcium carbonate 90% (an alkaline powder) at 55-70° C. to form granules. The granules were dried, and the dried granules were deagglomerated using a Comil® equipped with a 0.039″R (1,000 μm) screen. 0.32 kilos of magnesium stearate (a lubricant) and 0.08 kilos of colloidal silicon dioxide (a glidant) were added to the granules and a final mixing was performed. The final weight was thus about 39.8 kilos comprising about 7.96 kilos (20.0%) of diclofenac as the sodium salt.

### Example 2

The final mixture from Example 1 was filled into size 3 hard gelatin capsules at a net fill of 175 mg per capsule. Each capsule thus contained about 35 mg of diclofenac as diclofenac sodium.

### Example 3

Comparative bioavailability studies were performed using the capsules of Example 2 and ZORVOLEX™ capsules, both of 35 mg strength.

The results from a comparative bioavailability study in 35 subjects in the fasted state were: AUC_{T} ratio: 101.7% and Cₘₐₓ ratio 88.0%.

The results from a comparative bioavailability study in 36 subjects in the fed state were: AUC_{T} ratio: 100.4% and Cₘₐₓ ratio 91.1%.

The capsules of Example 2 are thus considered to provide equivalent AUC_{T} and Cₘₐₓ levels to those provided by ZORVOLEX™ capsules.

What is claimed is:

1. A process of manufacture of a solid dosage form for oral administration comprising the steps of:
(i) forming a solution by dissolving diclofenac and a basic sodium or potassium compound in a solvent comprising water, a volatile organic solvent, or a mixture thereof;
(ii) applying the solution to a solid substrate comprising one or more pharmaceutically acceptable excipients to form a mixture;
(iii) forming a dried mixture by evaporating the solvent from the mixture; and
(iv) further processing the dried mixture into a dosage form, optionally with one or more additional pharmaceutically acceptable excipients.

2. The process according to claim 1, wherein the basic sodium or potassium compound is sodium hydroxide or potassium hydroxide.

3. The process according to claim 1, wherein the solvent comprises water, an alcohol, or a mixture thereof.

4. The process according to claim 3, wherein the solvent comprises water.

5. The process according to claim 3, wherein the solvent comprises an alcohol.

6. The process of claim 1, wherein the further processing in step (iv) comprises granulating the dried mixture, optionally with one or more pharmaceutically acceptable excipients.

7. The process according to claim 1, wherein the substrate comprises a disintegrant.

8. The process according to claim 7, wherein the disintegrant comprises croscarmellose sodium.

9. The process according to claim 1, wherein substrate comprises a carbonate.

10. The process according to claim 9, wherein the carbonate is calcium carbonate.

11. The process according to claim 1, wherein the substrate comprises a mixture of a disintegrant and a carbonate.

12. The process according to claim 11, wherein the substrate comprises a mixture of croscarmellose sodium and calcium carbonate.

13. A solid dosage form for the oral administration of diclofenac prepared by:
(i) forming a solution by dissolving diclofenac and a basic sodium or potassium compound in a solvent comprising water, a volatile organic solvent, or a mixture thereof;
(ii) applying the solution to a solid substrate comprising one or more pharmaceutically acceptable excipients to form a mixture;
(iii) forming a dried mixture by evaporating the solvent from the mixture; and
(iv) further processing the dried mixture into a dosage form, optionally with one or more additional pharmaceutically acceptable excipients.

14. The solid dosage form of claim 13, wherein the dried mixture is granulated prior to mixing with one or more pharmaceutically acceptable excipients.

15. The solid dosage form of claim 14, wherein the substrate in the dried mixture comprises a disintegrant and a carbonate, and the dried mixture is mixed with a lubricant and a glidant.

16. The solid dosage form of claim 15, wherein the dosage form is a capsule filled with a dried mixture comprising diclofenac sodium and a substrate comprised of a mixture of croscarmellose sodium and calcium carbonate, and the dried mixture is mixed with magnesium stearate and colloidal silicon dioxide.

17. An orally-administrable solid dosage form of diclofenac comprising a substrate coated with a sodium or potassium diclofenac salt, and optionally also comprising, one or more pharmaceutically acceptable excipients.

18. The solid dosage form of claim 17, wherein the substrate is a disintegrant, a carbonate, or a mixture thereof.

19. The solid dosage form of claim 18, wherein the substrate is a mixture of croscarmellose sodium and calcium carbonate.

20. The solid dosage form of claim 17 comprising capsules filled with diclofenac sodium coated onto a solid substrate comprising a mixture of croscarmellose sodium and calcium carbonate, magnesium stearate and colloidal silicon dioxide.
